(19) United States
(12) Patent Application Publication
(10) Pub. No.: US 2020/0360590 A1
(43) Pub. Date: Nov. 19, 2020

(54) LOW MOLECULAR WEIGHT PECTIN COLUMN FOR APHERESIS

(71) Applicant: ISAAC ELIAZ, SANTA ROSA, CA (US)

(72) Inventor: ISAAC ELIAZ, SANTA ROSA, CA (US)

(21) Appl. No.: 16/740,460

(22) Filed: Jan. 12, 2020

Related U.S. Application Data
(60) Provisional application No. 62/849,084, filed on May 16, 2019.

(51) Int. Cl. A61M 1/34; B01J 20/32

(52) U.S. Cl. CPC A61M 1/3496; B01J 20/3274; A61M 1/3486

(57) ABSTRACT

An apheresis column for the treatment of mammals to reduce galectin-3 levels is set forth. The column features, as a stationary or adsorbent phase, pectin molecules, which naturally bind gal-3. Modified citrus pectin, having a molecular weight of about 40 kD or less, preferably about 25 kD or less, is a preferred adsorbent. The columns are disposable, and are used in the fashion for apheresis in general. Other targets, particularly including other galectins, as well as toxins, heavy metals and the like may also be withdrawn from the blood or plasma through this method. Gal-3 level reductions of 10%, 30% and even more may be achieved in a single treatment.

### INCORPORATION BY REFERENCE

This application is related to the use of pectin that has been modified from its natural form to exhibit a reduced molecular weight and less branching. This product, which typically has a molecular weight under forty thousand kD and more desirably under about twenty-five kD is made through processes that lead to a well characterized B-galactoside comprising pectin derivative. A preferred embodiment is often described as modified citurs pectin. The inventor is well recognized for innovation with the administration of modified citrus pectin—examples generally are directed to the administration—that is giving or providing—modified citrus pectin to individuals in need of specific treatments. Different inventions have taken various forms—from recognizing the need to reduce or control galectin-3 levels in patients identified as in need of that relief—such as patients needing to address inflammation or fibrosis, as set forth in U.S. Pat. No. 9,649,329; administration to mammals in need of immune system response improvement as discussed in U.S. Pat. No. 8,426,587; administration of modified citrus pectin for the purposes of binding of tumor emboli in U.S. Pat. No. 7,452,871; and administration for the purposes of binding or chelating of toxins and heavy metals as discussed in U.S. Pat. No. 6,274,566. These patents are all incorporated herein-by-reference.

### BACKGROUND OF THE INVENTION

### Field of the Invention

The present invention relates to a different use of modified pectin. As noted above, the ability of modified pectin—that is, pectin of a molecular weight below about 40,000 daltons, and preferably, even lower molecular weight around 15,000 daltons—to bind galectin-3 (gal-3 herein) is well demonstrated. As an effective agent in the control of inflammation, fibrosis, chronic heart disease, chronic and acute kidney disease, and a variety of other conditions, administration of modified citrus pectin (MCP herein) has been demonstrated repeatedly to address these conditions positively. Yet, gal-3 has been shown to be the mediator of a wide variety of conditions and disease states, beyond those referenced above. Control of gal-3 levels in the body would offer a non-medication-based treatment method that would benefit a wider variety of patients, not only by addressing specific mammalian patients, as noted in the patents discussed above but on a broader scale not confined to any one disease.

Pheresis, often referred to as apheresis or clinical apheresis, has been developed as ex vivo medical treatment to remove one or more elements of the blood by running withdrawn blood through a filter and returning the treated blood to the patient from which it was withdrawn. Although initially developed for the removal of units of the blood such as platelets or white blood cells, the inventor has developed apheresis as a blood treatment for, inter alia, withdrawal of gal-3 from mammalian blood. This technique is addressed in a variety of patents, some of which include U.S. Pat. No. 9,764,695 and U.S. Pat. No. 9,549,953 which provide for the selective withdrawal of gal-3 from the blood so processed. The gal-3 can be withdrawn from whole blood (apheresis) or from plasma (plasmapheresis). In these processes, the blood or plasma flows through a module provided with a unit that binds gal-3, such as an antibody that is specific therefor. The patents identified above are incorporated herein-by-reference.

### BACKGROUND OF THE INVENTION

This invention is premised on the recognition that an important aspect of health maintenance is control over gal-3 levels in the blood. While several antibodies specific for gal-3 are known (e.g., MA1-940 from ThermoFisher Scientific and AF1154 from R&D Systems), most known antibodies are for research only. Due to the possibility that the column contents used to remove gal-3 from the blood or plasma selectively might “leach” or escape into the body during the passage, a high level of certainty and safety is required before an antibody for binding gal-3 can be used in apheresis. This places extreme constraints on deliverability and cost.

Instead of an antibody binding medium, the Applicant has devised an apheresis column that relies on pectin, most preferably modified citrus pectin, to bind gal-3 in an apheresis procedure. Depending on the volume of blood treated, appreciable reductions in gal-3 blood levels can be achieved, using conventional modified citrus pectin that has been demonstrated safe for humans for ingestion and infusion for years (the product is characterized as “GRAS”—generally recognized as safe). Apheresis is a closed-circuit process where plasma is continually being restored to blood cells and entering back into the patient after being treated outside the patient. So the level of gal-3 reduction will vary with the procedure. However, the inventor has safely demonstrated gal-3 level reductions in excess of 30-40 percent of the gal-3 levels measured before apheresis is begun and at the completion of the apheresis treatment session.

Modified citrus pectin, or MCP, binds gal-3 naturally, without any further modification required. While this application focuses on modified citrus pectin, as popularly available on the market, in fact, a variety of modified pectins can be used. MCP is available, for example, from ecoNugenics, Inc. of Santa Rosa, Calif. under the trademark PectaSol-C® but other sources are known as well. Given its proven acceptability and uniform properties, this application is discussed in terms of MCP, which can be generally described as a powder substance having a molecular weight under about 40 thousand daltons, and desirably under 15 thousand daltons. It is water-soluble, so it must be bound to be included as the gal-3 attractant in an apheresis column such as that addressed herein. Thus, broadly phrased, the invention provides a column for apheresis, particularly adapted for the removal of gal-3 from the body. MCP binds to a variety of substances in the blood, but a principal target of this invention is gal-3.

Gal-3 is continually restored to circulation from body tissues in mammals, a process sometimes referred to as “shedding” gal-3. Thus, while the process disclosed and claimed herein can provide for a 100% removal of gal-3 from the blood collected to treat, gal-3 is released from the mammal's tissue into the blood. The more marked the condition, the more rapid the restoration of gal-3. For a healthy adult human, it may take as much as a week or ten days for gal-3 levels to return to pre-apheresis levels. In an individual with a high degree of inflammation, for example, patients diagnosed with sepsis, gal-3 levels may return in as little as 24-36 hours. Such patients may receive apheresis treatment daily or even continuously. Notwithstanding the tendency to “shed” gal-3 from tissues to blood, a healthy human patient can have the gal-3 level in their blood reduced by up to 50% or 50% or more after one apheresis treatment using the column of this invention with MCP as the adsorbent phase. Certainly, reductions of 40% or 30% or 25% can be easily achieved. (The values reported here are gal-3 levels in the blood post apheresis. If one considers only the volume of blood treated, before return to the mammal, often 100% of gal-3 present in blood can be apheresed in the column. At a minimum, 70%, 80% or 90% of the gal-3 present in the blood treated, before return to the body, is removed).

Even where the gal-3 values in the body following apheresis are as low as 25% of the normal values (a healthy adult may have in the range of 10-13 ng/ml gal-3 in their blood) are quickly restored following apheresis, the short term reduction of gal-3 levels is of benefit. (There is often confusion between serum and plasma levels. In general, serum values are values obtained after the blood has been clotted, while plasma refers to values where clotting has been prevented.) Marked reductions in gal-3 can quench the inflammation cascade in individuals and damp down the oncological pathways (reducing the fuel of the flames in many disease states). This gives the body's natural homeostatic regulators a chance to restore a healthy state. Even where the return to pre-apheresis levels is relatively rapid due to ongoing injury or stress (for example, acute kidney injury or chronic kidney injury) progress toward homeostasis benefits the body. With multiple treatments over time, it is anticipated that inflammation will decrease and disease progression slow or reverse. Given that gal-3 levels in the blood are relatively low, even a 30%, 40% or 50% decrease realized through this invention provides a significant clinical effect.

This application focuses on a very narrow window of use of MCP. MCP is well known as a supplement for a wide variety of purposes but has been shown to improve mammalian immune responses, reduce inflammation and fibrosis, improve the treatment of chronic heart disease, and other indications. This application focuses on the use of MCP as a binding source to remove gal-3 in an apheresis process. Accordingly, the properties of MCP as a supplement are not further addressed herein but can be found in a wide variety of journals and the patents listed above. This application begins with a description of the column itself—the apheresis column with MCP or other pectins as a medium that adsorbs gal-3 from the bloodstream during passage through the column, and may then be safely returned to the mammalian patient. The possible use of a column with MCP to reduce gal-3 levels in the body through apheresis is briefly described in US 2016/0317734. A general description appears at about [0032]. This application begins where that brief reference ends—providing a far more detailed and specific disclosure of how to make and use the column. Accordingly, below, this application proceeds with a description of how the apheresis column is prepared, and then how it may be used. This column, while it may be used to adsorb a number of products from the blood, as discussed below, is specifically designed to promote safe and effective removal of significant amounts of gal-3. In fact, MCP binds to all known mammalian galectins. Gal-3 is, however, the only known chimeric galectin, which with the unique ability to form pentamers, and so maybe bound in significant amounts.

### DESCRIPTION OF THE FIGURES

This application makes use of the ability of pectin, particularly modified citrus pectin, which has been proven safe and well tolerated in mammals, to bind gal-3. Thus, the molecular character or structure of both MCP and gal-3 are of importance to this invention.

FIG. 1 illustrates the molecular structure of MCP.

FIG. 2 illustrates the molecular structure of galectins in general, and gal-3 in particular, which may adopt a monomeric, dimeric or chimeric molecular structure.

### DETAILED DESCRIPTION OF THE INVENTION

Apheresis is a well-known procedure that was developed in the early 1970s. Since that time, the use of apheresis has become widespread. This application simply applies this wide knowledge of extracorporeal practice to the use of a specific apheresis column—one using modified pectin, preferably MCP. There is an extensive collection of articles and scientific discussion of apheresis technology in de Sousa G, and Seghatchian J. Transfusion and Apheresis Science. 2006 February;34(1):107-23. Although much has been developed since that time as well, the invention here addresses a new column, rather than a specific new use. With the new column, of course, goes a new specific goal—the reduction of gal-3 in the blood by apheresis—but other than specific adaptations due to the use of modified pectin or MCP in particular, the general practice remains the same. Thus, this discussion begins with a consideration of how the column is prepared.

### HOW THE COLUMN AND MATERIALS ARE MADE

Covalent immobilization of low molecular mass pectin using EDC [1-Ethyl-3-(3-dimethylaminopropyl)carbodiimide HCl]/NHS (N-hydroxysuccinimide esters) primary amine linking chemistry [link through a galUA (galacturonic acid) carboxylate on MCP] to agarose beads bearing an amine group. The carboxyl-containing (—COOH) galUAs to a porous, beaded agarose resin for use in affinity purification procedures using extracorporeal apheresis of plasma or whole blood. The crosslinked beaded agarose is activated with diamino-dipropylamine (DADPA) to contain long spacer arms, each with a primary amine at the end. When incubated with the resin and the carbodiimide crosslinker EDC, carboxyl-containing molecules become permanently attached to the support by stable amide bonds.

Additional details on the immobilization chemistry for carboxyl-containing molecules required to prepared the column begins with the effective crosslinking agents. EDC crosslinker reacts with carboxylates to create an amine-reactive intermediate, resulting in a covalent attachment to the DADPA-activated Resin General References (for EDC coupling chemistry), Gilles, M. A. et al., Anal Biochem 1990;184:244-8; Grabarek, Z., and Gergely, J. Anal Biochem 1990;185:131-5; Williams, A., and Ibrahim, I. A. J Am Chem Soc 1981;103:7090-5.

Prior art characterizations of MCP focus on water-soluble supplements intended for oral, or, where appropriate, IV administration. Other routes of administration, such as via rectal or vaginal suppositories, may be available. The invention of this application is, however, directed to a stable, chemically modified version of MCP or other pectin or similar which is characterized as follows.

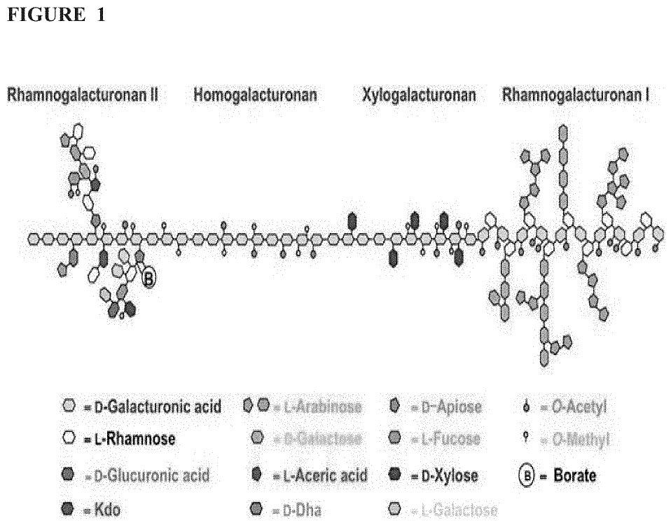

Pectin's ability to bind a variety of molecules, and galectins, including gal-3 in particular, are key to the effective use of the column of this invention. The structure of low molecular weight pectin is illustrated in FIG. 1 of this application. Pectin consists of four different types of polysaccharides, and their structures are shown. (Abbreviations: Kdo, 3-Deoxy-d-manno-2-octulosonic acid; DHA, 3-deoxy-d-lyxo-2-heptulosaric acid.) HG and RGI are much more abundant than the other components.

Pectin is a complex plant polysaccharide consisting of homogalacturonan (HG), which is partially methyl esterified; rhamnogalacturonan I (RGI), consisting of alternating rhamnose and galacturonic acid residues with arabinan, galactan and/or arabinogalactan attached to the rhamnose residues; rhamnogalacturonan II (RGII), with a homogalacturonan backbone and complex branches containing neutral and acidic sugars including 2-keto-3-deoxyoctonic acid (KDO); and xylogalacturonan, with xylose attached to some of the galacturonic acid residues.

The ratio between HG, XGA, RGI, and RGII is variable, but typically HG is the most abundant polysaccharide, constituting about 65% of the pectin, while RGI constitutes 20% to 35%. XGA and RGII, each representing 10% or less. The different pectic polysaccharides are not separate molecules but covalently linked domains. Unbranched homopolymer chains of α-1,4-linked d-GalUA are described as HG. The backbone of GalUA residues can be substituted at various positions with other sugar moieties, including Xyl (XGA) and apiofuranose (apiogalacturonan). In XGA, a single Xyl is attached to the O-3 position of some GalUA residues. Additional Xyl residues can be attached to the first Xyl with β-1,4 linkage.

The size and composition of MCP are consistent with reduced molecular weight and debranched pectin consisting of a homogalacturonan backbone with little methyl esterification. The galacturonic acid content of MCP analyzed by USDA-ARS pectin scientist (Eliaz et al., Phytother Res. 2006 October;20(10):859-64) was 74.6% (±3.8), the degree of esterification was 3.8%, the KDO content was 0.46% (±0.0) and the total neutral sugar carbohydrate content was 10.1% (±0.7). The neutral monosaccharides in MCP were consistent with the neutral sugar-rich side chains in the rhamnogalacturonan region of pectin (Ridley et al., Phytochemistry. 2001 July;57(6):929-67.).

### DETAILED DESCRIPTION REGARDING USE OF THE COLUMN

The blood or plasma of a person or other mammal is passed through the affinity column to separate out lectins, such as galectins in the circulation before returning the remainder back to the circulation. It is thus an extracorporeal therapy. Galectins are carbohydrate-binding proteins that are involved in many physiological functions, such as inflammation, immune responses, cell migration, autophagy, and signaling. They are also linked to diseases such as fibrosis, cancer and heart disease. The apheresis procedure lasts several hours depending on a multiple of variables such as patient blood volume, flow rate, and binding capacity, final column size and dimensions.

It is difficult to estimate the consequence of treatment in a column of the invention due to the fact that as the patient is treated, additional gal-3 is shed by the patient's tissues into the blood. For purposes of analysis, set a standard plasma volume, in a 70 KG adult, as about 3,000 ml (a close estimate). Apheresis using this column has been demonstrated to reduce nearly 100% of the gal-3 in the plasma being treated prior to treatment. In a chronic kidney disease patient with end-stage renal disease, up to 70 ng/ml gal-3, or about 210 micrograms, may be pulled from the blood being treated. It is important to understand that due to gal-3 replacement from tissues, the kinetics are such that in two plasma volumes, you remove only 80%, meaning 160 micrograms.

The principal target to be bound ex vivo through the use of the pectin-based column of this invention is galectin-3. As noted, gal-3 is the one galectin that appears not only as a monomer and dimer, but a chimeric form, sometimes considered a pentamer, as well. Gal-3 is not the only potential target for binding by MCP, however. Pectins, in general, can bind other environmental toxins in addition to the heavy metals mentioned, such as DDT (Zhan et al., Environment Intern 2019 September;130:104861). Evidence demonstrates the ability of pectin, and MCP in particular, to bind dioxin and dioxin-like toxins (Aozasa et al., Chemosphere 2001 October;45(2):195-200), radioactive isotopes (Nesterenko et al., Swiss Med Wkly, 2004 Jan. 10;134(1-2):24-7), and there is evidence that at least MCP can trap mycotoxins (Tamura et al., Carbohydr Polym. 2013 Apr. 2;93(2):747-52). Pectin binds to cholesterol in the gastrointestinal tract and slows glucose absorption by trapping carbohydrates. Thus, this invention is directed to and explains in detail the use of pectins, such as MCP, in a column to bind gal-3, and thereby reduce gal-3 levels in a patient through apheresis. The same apheresis treatment may be used to address other targeted or selective removal at the same time, where conditions warrant.

To remove specific blood components such as lectins in general (partners that bind particular carbohydrate structures), and specifically galectins [with an affinity to β-galactose-containing glycoconjugates and share primary structural homology in their carbohydrate-recognition domains (CRDs)], as well as other carbohydrate bind proteins found in the circulation. In general, other components found in the blood which have previously been established as being well-bound by pectin, with MCP as an example, maybe targeted for removal by this process. Thus, in addition to gal-3 in particular, and lectins more generally, components associated with morbidity and mortality, including oxidized lipids, positively charged heavy metals, toxins and the like may be effectively removed using the column of the invention.

The column of this invention, specifically, an apheresis column comprising pectin or another polyuronide, preferably modified citrus pectin having a molecular weight of less than 40 kD, is used in a fashion not dissimilar from other apheresis columns. This invention allows apheresis to be performed on either whole blood or plasma. Whole blood may offer advantages, which can be achieved using larger columns with longer and larger pathways. Specifically, blood is withdrawn from a patient. Blood may be separated, often by centrifugation, into plasma and cell components. If treating plasma only, the components are returned to the body. The plasma is directed under low pressure through a tube or module which provides a pathway for the plasma through the adsorbent phase. A prior art adsorbent phase use in apheresis for removal of low-density lipids, or liposorption, uses dextran sulfate as the adsorbent phase. The column of the claimed invention is used in a very similar manner, but offers a different adsorbent—pectin—to remove a different blood/plasma component—gal-3. The apheresis treatment can be continuous or discontinuous, depending on the pump and other apparatus available.

As noted, this invention is focused on the ability to remove gal-3 and lower gal-3 serum and blood levels. Gal-3 mediates a wide variety of diseases and complications. Among the most profoundly impacted are kidney patients, including those suffering from chronic kidney disease (CKD) and acute kidney disease (AM). Treatments generally adopted for such patients may be advantageously administered with or immediately after apheresis to reduce gal-3 levels. Among the more well-known and generalized therapies are the administration of diuretics, and calcium and insulin to avoid dangerous increases in blood potassium levels. Vasodilators, such as fenoldopam and minoxidil, may be used to reduce the need for renal replacement therapy and lower the mortality rate in patients with AM. ACE inhibitors and angiotensin receptor blockers (ARBs) may be used as well as anti-inflammatory and kidney protective compounds with greater effectiveness given reduced Gal-3 levels and the reduced levels of inflammation and fibrosis formation resulting. Another group of patients fundamentally impacted by gal-3 levels and the conditions mediated by gal-3 are cancer patients. A wide variety of tumor-based cancers are mediated, at least in part, by gal-3 levels.

Other more targeted therapies may be made more effective by being practiced together with Gal-3 apheresis. AM occurs in about fifty percent of patients with septic shock. Traditional treatments for septic shock, such as vasopressors, inotropic (e.g., clonidine) and the like are advantageously combined in acute patients with Gal-3 apheresis. Angiopoietin levels are closely associated with the pathogenesis of vascular permeability, and established angiopoietin disciplines may be more effective at low Gal-3 systemic levels. Ebihara et al., Ther Apher Dial. 2016;20(4):368-75. As a general proposition, most of the therapeutics used to try to slow the progress of kidney damage and disease will be more effective following and/or combined with Gal-3 apheresis, as it will reduce the tendency of fibrotic growth to block the damaged tissue from the pharmaceutical agents systemically administered. To this end, conventionally used cardiac, renal and circulatory medications may be employed to further support the patient together with the selective removal of Gal-3. In addition to the therapeutics discussed above, conventionally used therapeutics include beta-blockers, calcium channel blockers, direct renin inhibitors, alpha-blockers and alpha 2 agonists (e.g., clonidine). Erythropoietin (rhEPO) and iron replacement therapy may be effective, as well as other supplements, such as vitamins and the like. To further increase effectiveness, such agents can be administered to the plasma or blood being returned to the patient after Gal-3 removal. In this respect, the enhancement of traditional dialysis by the use of gal-3 selective removal through apheresis may greatly reduce inflammation and fibrotic kidney injury. This will allow a significant increase in the percentage of patients having a complete recovery of kidney function for both short and long term care solutions.

The reduction of gal-3 levels is the specific goal of this invention. Gal-3 is ubiquitously expressed in the heart, the kidney, blood vessels, and macrophages play a vital role in tissue fibrosis, immunity, and the inflammatory response. Furthermore, extracellular gal-3 cross-links glycoconjugates and form lattices. Formation of galectin/glycoconjugate lattices on the plasma membrane has been observed to influence the expression time, localization, and activity of several cell surface receptors, thus affecting numerous biological functions such as cell signaling, cell migration, and cell adherence. MCP, as administered, disrupts the lattice formation with positive consequences for several physiological processes related to immune responses and inflammation, as well as pathological conditions such as fibrosis, cancer, and heart disease. This invention takes advantage of the binding properties of low molecular weight pectin, generally, and MCP specifically, to reduce gal-3 levels, ex vivo, which makes possible a wide variety of applications.

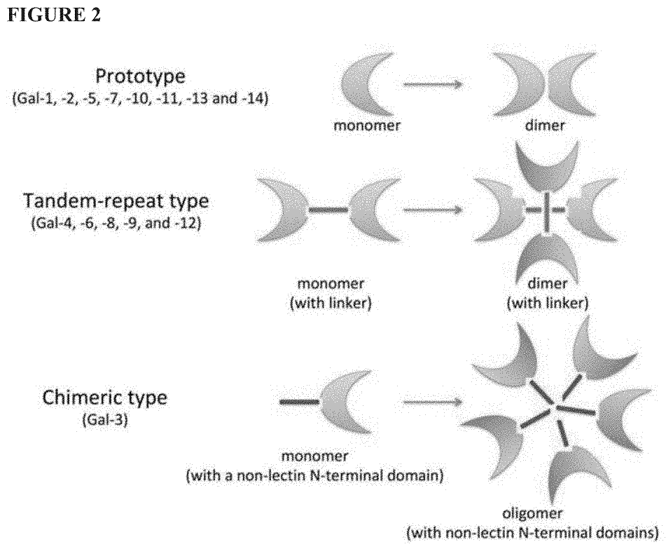

MCP provides multiple carbohydrate sites for binding up the receptor CRD regions of gal-3 antagonizing its proinflammatory and tumorigenesis effects. The binding affinity of the galectin chain is proportional to its length up to 4 gal residues and mostly unchanged after that. MCP can bind to all galectin, but at different affinities. The strength of ligand binding is determined by several factors: The multivalency of both ligand and the galectin, the length of the carbohydrate, and the mode of presentation of ligand to the CRD. Different galectins have distinct binding specificities for binding oligosaccharides. The only chimeric type, gal-3, with its ability to oligomerize to a pentamer, provides a particularly apt target for binding by MCP, offering a wide array of carbohydrate ligands on its side chains and backbone makes for high-affinity selective binding. With gal-3 being overexpressed into the circulation in the medical conditions mentioned above, the most prevalent extracellular galectin found in circulation, gal-3 would be the predominant target and most effectively removed agent using the column of this invention. The molecular characteristics of gal-3 demonstrating the particular suitability of the column of this invention, are illustrated in FIG. 2 of this Application.

What is claimed is:

1. An apheresis column for the ex vivo treatment of a mammal, comprising a column with a pathway through which at least a portion of the mammal's blood is directed, wherein said pathway is provided with units of immobilized low molecular weight pectin to which said blood is exposed, wherein at least galectin-3 in said blood is bound by said pectin molecules in said column, after which said blood is returned to said mammal, wherein following apheresis, galectin-3 levels in the mammal are reduced by at least 10%.

2. The column of claim 1, wherein said blood is separated into plasma and non-plasma components prior to entering said pathway, and wherein only said plasma is passed through said pathway.

3. The column of claim 1, wherein the level of galectin-3 in said patient following apheresis treatment in said column is reduced by at least 30 percent.

4. The column of claim 1, wherein said immobilized pectin is comprised of modified citrus pectin having a molecular weight less than 40 kD.

5. The column of claim 4, wherein said modified citrus pectin is of a molecular weight less than 25 kD.

6. A process for the ex vivo removal of galectin-3 from the blood of a mammal, comprising:
directing said blood to a column comprising a pathway provided with units of immobilized low molecular weight pectin to which said blood is exposed, wherein at least galectin-3 in said blood is bound by said pectin units in said column,
thereafter returning said blood to said mammal, whereby at least 10% of the galectin-3 in the blood of said mammal is removed.

7. The process of claim 6, wherein said blood is separated into cellular components and plasma prior to being directed to said column, and wherein only said plasma is directed to said column.

8. The process of claim 7, wherein at least 80% of the galectin-3 in the blood directed to said column is removed.

9. The process of claim 6, wherein, immediately following said process, the mammal so treated exhibits a galectin-3 level at least ten percent lower than the galectin-3 level of the blood of said mammal immediately prior to that process.

10. The process of claim 9, wherein immediately following said process, the mammal so treated exhibits a galectin-3 level at least 30% lower than the galectin-3 level of said mammal immediately prior to that process.

11. The process of claim 6, wherein said process is accompanied by treatment of said mammal with one or more agents which are therapeutically more effective in the presence of reduced levels of galectin-3.

12. The method of claim 6, wherein said mammal is a human being.
